(19) United States
(12) Patent Application Publication
(10) Pub. No.: US 2006/0100672 A1
(43) Pub. Date: May 11, 2006

(54) Method and system of matching information from cochlear implants in two ears

(76) Inventor: Leonid Michael Litvak, Los Angeles, CA (US)

Correspondence Address: FISH & RICHARDSON P.C., P.O. BOX 1022, MINNEAPOLIS, MN, 55440-1022, US

(21) Appl. No.: 10/982,371

(22) Filed: Nov. 5, 2004

(51) Int. Cl. A61N 1/08

(52) U.S. Cl. 607/57

(57) ABSTRACT

Disclosed are systems and methods for matching pitch information between bilateral cochlear implants in order to maximize a patient's listening experience. The system permits an electrode array of a first cochlear implant to be pitch matched to an electrode array of a second cochlear implant system by utilizing virtual electrodes, which enable cochlear stimulation at a location in between physical electrodes on the electrode array. At least one electrode of the first electrode array is mapped to a virtual electrode of the second electrode array.

### TECHNICAL FIELD

This disclosure relates to systems and methods for stimulating the cochlea, and more particularly to systems and methods for matching sound information from a cochlear implant in one ear to a cochlear implant in another ear.

### BACKGROUND

Prior to the past several decades, scientists generally believed that it was impossible to restore hearing to the deaf. However, scientists have had increasing success in restoring normal hearing to the deaf through electrical stimulation of the auditory nerve. The initial attempts to restore hearing were not very successful, as patients were unable to understand speech. However, as scientists developed different techniques for delivering electrical stimuli to the auditory nerve, the auditory sensations elicited by electrical stimulation gradually came closer to sounding more like normal speech. The electrical stimulation is implemented through a prosthetic device, called a cochlear implant, that is implanted in the inner ear to restore partial hearing to profoundly deaf people.

Such cochlear implants generally employ an electrode array that is inserted in a cochlear duct, usually the scala tympani. One or more electrodes of the array selectively stimulate different auditory nerves at different places in the cochlea based on the pitch of a received sound signal. Within the cochlea, there are two main cues that convey “pitch” (frequency) information to the patient. These are (1) the place or location of stimulation along the length of a cochlear duct and (2) the temporal structure of the stimulating waveform. In the cochlea, sound frequencies are mapped to a “place” (i.e., a specific location along) in the cochlea, generally from low to high sound frequencies mapped from the apical to basilar direction. The electrode array is fitted to the patient to arrive at a mapping scheme such that electrodes near the base of the cochlea are stimulated with high frequency signals, while electrodes near the apex are stimulated with low frequency signals

Mapping an electrode array in a cochlear duct to the correct audio frequencies is complicated by the differences in an individual's anatomy. In addition, the final implanted position of the electrode array is variable and also lends an arbitrariness to a mapping scheme between an electrode contact and a perceived sound frequency. Thus, an optimal fitting map between an electrode contact and a sound frequency can only be roughly guessed at the outset for each individual. The initial guess is almost always inaccurate for that individual.

In addition, the position of each electrode is not very precise. That is, there are only a limited number of electrodes, e.g., numbering about 16 to 24 electrodes, spread along the length of the electrode array inserted into one of the spiraling ducts of the cochlea. Hence, accurately mapping to a “place” within the cochlea can be difficult, as the mapping is limited by the resolution of the discretely placed electrodes.

### SUMMARY

The uncertainties in electrode mapping are compounded with the use of bilateral implants. Early research indicates that cochlear implant patients will benefit from additional synchronized and processed speech information conveyed to the brain via both the right and left auditory nerve pathways using bilateral implants. However, if the electrode array within the two cochleas of the patient are not properly matched to one another in terms of pitch, the patient's hearing between the two ears may be adversely affected. In particular, research studies show that certain binaural cues are only accessible if information is presented to pitch-matched electrodes. Thus, it is beneficial for bilateral implants to be pitch matched such that, for the same sound, a pitch generated along one implant matches the pitch generated by the other implant. Disclosed are methods and systems for matching pitch information between bilateral cochlear implants. Conventional methods that use only physical electrodes only afford limited precision. The concept of virtual electrodes, described in detail below, permits greater precision.

In one aspect, there is shown a method of pitch matching a first cochlear implant to a second cochlear implant. A first stimulus current is applied to a first electrode of a first multi-electrode array implanted in a first cochlea of a user to generate a first tone relative to a first ear of the user. A second stimulus current is also applied to a second electrode of a second multi-electrode array in a second cochlea of the user to generate a second tone relative to a second ear of the user. Feedback is then obtained as to whether the first tone is higher, lower, or the same as the second tone. If it is determined that the first tone does not match the second tone, a stimulus current is applied to a virtual electrode of the second multi-electrode array such that stimulation of the virtual electrode generates a third tone that matches the first tone. A position of the first electrode of the first multi-electrode array is then mapped to a position of the virtual electrode of the second multi-electrode array. Mapping the first electrode of the first multi-electrode array to the virtual electrode of the second multi-electrode array can comprise, for example, allocating a frequency of a received sound signal to both the first electrode and to the virtual electrode.

In another aspect, there is shown a method of matching a first cochlear implant to a second cochlear implant. A stimulus current is applied to a first electrode of a first multi-electrode array implanted in a first cochlea of a user to generate a first tone in a first ear of the user. The cochlear position is determined for a first virtual electrode of a second multi-electrode array implanted in a second cochlea of the user. Stimulation of the virtual electrode results in a second tone in the second ear of the user that matches the first tone. The first electrode is then mapped to the first virtual electrode.

In another aspect, there is shown a bilateral cochlear stimulation system. The system comprises a first cochlear implant system comprising first multi-electrode array having a first plurality of electrodes configured for placement in first cochlear duct of a patient and a second cochlear implant system comprising a second multi-electrode array having a second plurality of electrodes configured for placement in a second cochlear duct of a patient. The second multi-electrode array is configured to implement virtual electrodes. At least a first electrode of the first multi-electrode array is mapped to at least one virtual electrode of the second multi-electrode array.

The details of one or more embodiments are set forth in the accompanying drawings and the description below. Other features and advantages will be apparent from the description and drawings, and from the claims.

### DESCRIPTION OF DRAWINGS

The features and advantages will be more apparent from the following more particular description thereof, presented in conjunction with the following drawings, wherein:

FIG. 1 is a current stimulation waveform that defines the stimulation rate (1/T) and biphasic pulse width (PW) associated with electrical stimuli, as those terms are commonly used in the neurostimulation art;

FIGS. 2A and 2B, respectively, show a cochlear implant system and a partial functional block diagram of the cochlear stimulation system, which system is capable of providing high rate pulsatile electrical stimuli and virtual electrodes

FIG. 3A schematically illustrates the locations of applied stimuli within a duct of the cochlea, without the benefit of virtual electrodes;

FIG. 3B schematically illustrates the locations of applied stimuli within a duct of the cochlea or other implanted location, with the benefit of virtual electrodes;

FIG. 4 presents a flow diagram that outlines one embodiment of the present method for matching information from cochlear implants in two ears;

FIG. 5 shows a system configuration that can be used to synchronize bilateral cochlear implant systems; and

FIG. 6 shows a system configuration that can be used to synchronize bilateral cochlear implant systems.

Like reference symbols in the various drawings indicate like elements.

### DETAILED DESCRIPTION

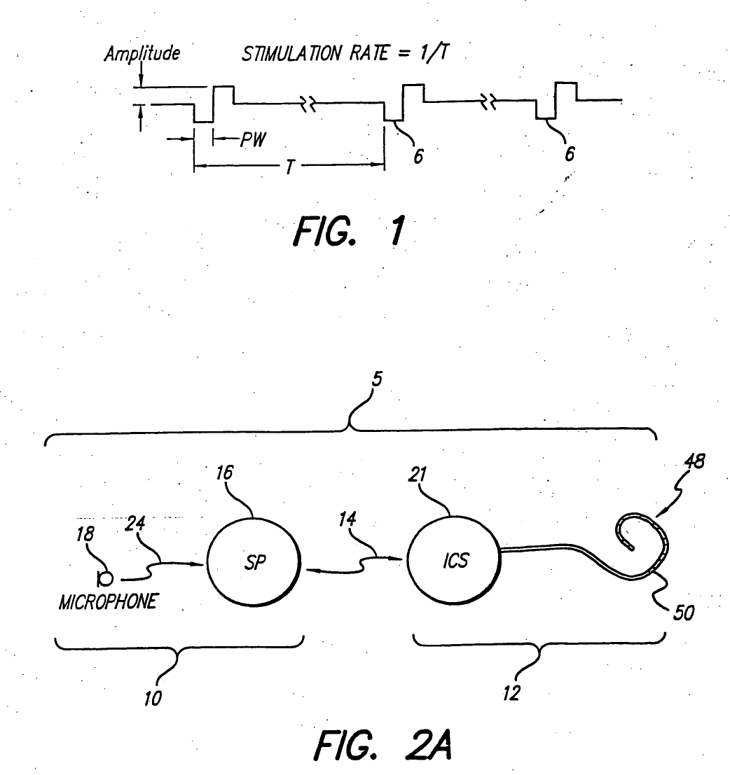

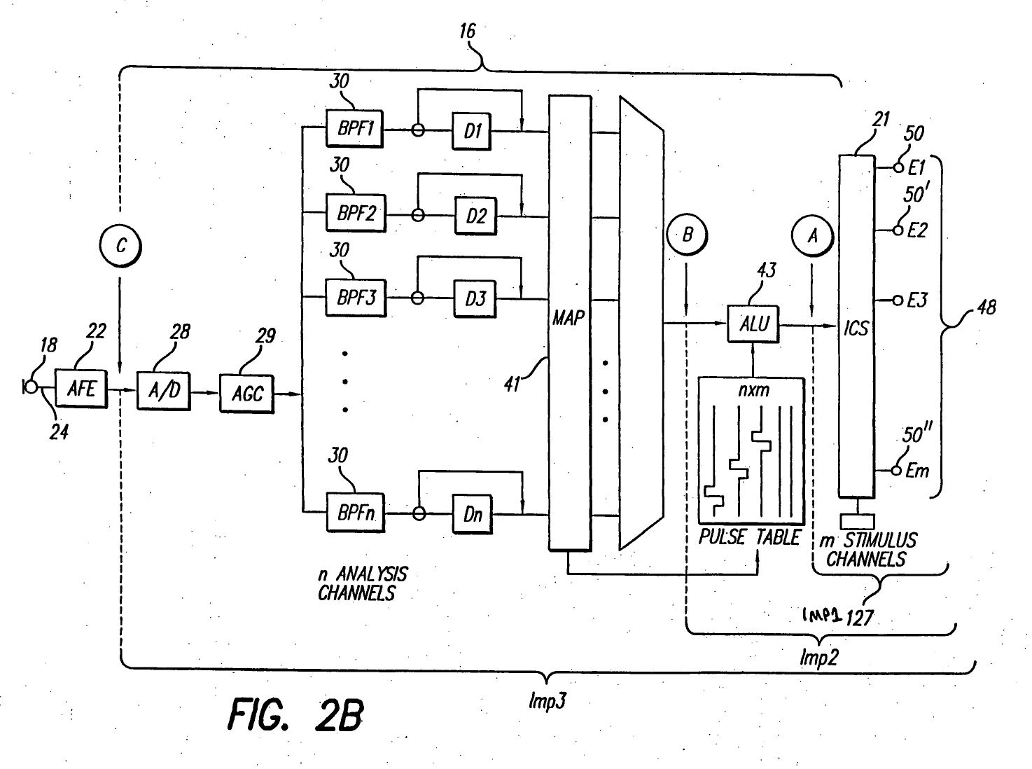

Disclosed are devices and methods for matching information between cochlear implants in two ears of a patient. It will be helpful to first provide an overview of the structure and functionality of an exemplary cochlear implant system. This overview is provided below in connection with the description of FIGS. 1, 2A and 2B. It should be appreciated that the following description is exemplary and that the device and methods described herein can be used with other types and other configurations of cochlear implant systems.

FIG. 1 shows a waveform diagram of a biphasic pulse train. The figure defines stimulation rate (1/T), pulse width (PW) and pulse amplitude as those terms are commonly used in connection with a neurostimulator device, such as a cochlear implant, a spinal cord stimulator (SCS), a deep brain stimulator (DBS), or other neural stimulator. All such systems commonly generate biphasic pulses 6 of the type shown in FIG. 1 in order to deliver stimulation to tissue.

A “biphasic” pulse 6 consists of two pulses: a first pulse of one polarity having a specified magnitude, followed immediately or after a very short delay by a second pulse of the opposite polarity, although possibly of different duration and amplitude, such that the total charge of the first pulse equals the total charge of the second pulse. It is thought that such charge-balancing can prevent damage to stimulated tissue and prevent electrode corrosion. For multichannel cochlear stimulators, it is common to apply a high rate biphasic stimulation pulse train to each of the pairs of electrodes in the implant (described below) in accordance with a selected strategy and modulate the pulse amplitude of the pulse train as a function of information contained within the sensed acoustic signal.

FIG. 2A shows a cochlear stimulation system 5 that includes a speech processor portion 10 and a cochlear stimulation portion 12. The speech processor portion 10 includes a speech processor (SP) 16 and a microphone 18. The microphone 18 may be connected directly to the SP 16 or coupled to the SP 16 through an appropriate communication link 24. The cochlear stimulation portion 12 includes an implantable cochlear stimulator (ICS) 21 and an electrode array 48. The electrode array 48 is adapted to be inserted within the cochlea of a patient. The array 48 includes a plurality of electrodes 50, e.g., sixteen electrodes, spaced along the array length and which electrodes are selectively connected to the ICS 21. The electrode array 48 may be substantially as shown and described in U.S. Pat. Nos. 4,819,647 or 6,129,753, both patents incorporated herein by reference. Electronic circuitry within the ICS 21 allows a specified stimulation current to be applied to selected pairs or groups of the individual electrodes included within the electrode array 48 in accordance with a specified stimulation pattern defined by the SP 16.

The ICS 21 and the SP 16 are shown in FIG. 2A as being linked together electronically through a suitable data or communications link 14. In some cochlear implant systems, the SP 16 and microphone 18 comprise the external portion of the cochlear implant system and the ICS 21 and electrode array 48 comprise the implantable portion of the system. Thus, the data link 14 is a transcutaneous (through the skin) data link that allows power and control signals to be sent from the SP 16 to the ICS 21. In some embodiments, data and status signals may also be sent from the ICS 21 to the SP 16.

Certain portions of the cochlear stimulation system 5 can be contained in a behind the ear (BTE) unit that is positioned at or near the patient's ear. For example, the BTE unit can include the SP 16 and a battery module, which are coupled to a corresponding ICS 21 and an electrode array 48. A pair of BTE units and corresponding implants can be communicatively linked via a Bionet and synchronized to enable bilateral speech information conveyed to the brain via both the right and left auditory nerve pathways. A system for allowing bilateral implant systems to be networked together is described in co-pending U.S. patent application Ser. No. 10/218,615, entitled “Bionet for Bilateral Cochlear Implant Systems”, which is incorporated herein by reference in its entirety and assigned to the same assignee as the instant application. The Bionet system uses an adapter module that allows two BTE units to be synchronized both temporally and tonotopically in order to maximize a patient's listening experience.

FIG. 2B shows a partial block diagram of one embodiment of a cochlear implant system capable of providing a high pusatile stimulation pattern and virtual electrodes, which are described below. At least certain portions of the SP 16 can be included within the implantable portion of the overall cochlear implant system, while other portions of the SP 16 can remain in the external portion of the system. In general, at least the microphone 18 and associated analog front end (AFE) circuitry 22 can be part of the external portion of the system and at least the ICS 21 and electrode array 48 can be part of the implantable portion of the system. As used herein, the term “external” means not implanted under the skin or residing within the inner ear. However, the term “external” can also mean residing within the outer ear, residing within the ear canal or being located within the middle ear.

Typically, where a transcutaneous data link must be established between the external portion and implantable portions of the system, such link is implemented by using an internal antenna coil within the implantable portion, and an external antenna coil within the external portion. In operation, the external antenna coil is aligned over the location where the internal antenna coil is implanted, allowing such coils to be inductively coupled to each other, thereby allowing data (e.g., the magnitude and polarity of a sensed acoustic signals) and power to be transmitted from the external portion to the implantable portion. Note, in other embodiments, both the SP 16 and the ICS 21 may be implanted within the patient, either in the same housing or in separate housings. If in the same housing, the link 14 may be implemented with a direct wire connection within such housing. If in separate housings, as described, e.g., in U.S. Pat. No. 6,067,474, incorporated herein by reference, the link 14 may be an inductive link using a coil or a wire loop coupled to the respective parts.

The microphone 18 senses sound waves and converts such sound waves to corresponding electrical signals and thus functions as an acoustic transducer. The electrical signals are sent to the SP 16 over a suitable electrical or other link 24. The SP 16 processes these converted acoustic signals in accordance with a selected speech processing strategy to generate appropriate control signals for controlling the ICS 21. Such control signals specify or define the polarity, magnitude, location (which electrode pair or electrode group receive the stimulation current), and timing (when the stimulation current is applied to the electrode pair) of the stimulation current that is generated by the ICS. Such control signals thus combine to produce a desired spatio-temporal pattern of electrical stimuli in accordance with a desired speech processing strategy.

A speech processing strategy is used, among other reasons, to condition the magnitude and polarity of the stimulation current applied to the implanted electrodes of the electrode array 48. Such speech processing strategy involves defining a pattern of stimulation waveforms that are to be applied to the electrodes as controlled electrical currents.

FIG. 2B depicts the functions that are carried out by the SP 16 and the ICS 21. It should be appreciated that the functions shown in FIG. 2B (dividing the incoming signal into frequency bands and independently processing each band) are representative of just one type of signal processing strategy that may be employed. Other signal processing strategies could just as easily be used to process the incoming acoustical signal. A description of the functional block diagram of the cochlear implant shown in FIG. 2B is found in U.S. Pat. No. 6,219,580, incorporated herein by reference. The system and method described herein may be used with other cochlear systems other than the system shown in FIG. 2B, which system is not intended to be limiting.

The cochlear implant functionally shown in FIG. 2B provides n analysis channels that may be mapped to one or more stimulus channels. That is, after the incoming sound signal is received through the microphone 18 and the analog front end circuitry (AFE) 22, the signal can be digitized in an analog to digital (A/D) converter 28 and then subjected to appropriate gain control (which may include compression) in an automatic gain control (AGC) unit 29. After appropriate gain control, the signal can be divided into n analysis channels 30, each of which includes at least one bandpass filter, BPFn, centered at a selected frequency. The signal present in each analysis channel 30 is processed as described more fully in the 6,219,580 patent, or as is appropriate, using other signal processing techniques and the signals from each analysis channel may then be mapped, using mapping function 41, so that an appropriate stimulus current of a desired amplitude and timing may be applied through a selected stimulus channel to stimulate the auditory nerve.

The exemplary system of FIG. 2B provides a plurality of analysis channels, n, wherein the incoming signal is analyzed. The information contained in these n analysis channels is then appropriately processed, compressed and mapped in order to control the actual stimulus patterns that are applied to the user by the ICS 21 and its associated electrode array 48.

The electrode array 48 includes a plurality of electrode contacts 50, 50′, 50″ and labeled as, E1, E2 . . . Em, respectively, which are connected through appropriate conductors to respective current generators or pulse generators within the ICS. Through these plurality of electrode contacts, a plurality of stimulus channels 127, e.g., m stimulus channels, may exist through which individual electrical stimuli can be applied at m different stimulation sites within the patient's cochlea or other tissue stimulation site.

It can be common to use a one-to-one mapping scheme between the n analysis channels and the m stimulus channels 127 that are directly linked to m electrodes 50, 50′, 50″, such that n analysis channels=m electrodes. In such a case, the signal resulting from analysis in the first analysis channel may be mapped, using appropriate mapping circuitry 41 or equivalent, to the first stimulation channel via a first map link, resulting in a first cochlear stimulation place (or first electrode). Similarly, the signal resulting from analysis in the second analysis channel of the SP may be mapped to a second stimulation channel via a second map link, resulting in a second cochlear stimulation place, and so on.

In some instances, a different mapping scheme may prove to be beneficial to the patient. For example, assume that n is not equal to m (n, for example, could be at least 20 or as high as 32, while m may be no greater than sixteen, e.g., 8 to 16). The signal resulting from analysis in the first analysis channel may be mapped, using appropriate mapping circuitry 41 or equivalent, to the first stimulation channel via a first map link, resulting in a first stimulation site (or first area of neural excitation). Similarly, the signal resulting from analysis in the second analysis channel of the SP may be mapped to the second stimulation channel via a second map link, resulting in a second stimulation site. Also, the signal resulting from analysis in the second analysis channel may be jointly mapped to the first and second stimulation channels via a joint map link. This joint link results in a stimulation site that is somewhere in between the first and second stimulation sites.

The “in-between” site at which a stimulus is applied may be referred to as a “stimulation site” produced by a virtual electrode. Advantageously, this capability of using different mapping schemes between n SP analysis channels and m ICS stimulation channels to thereby produce a large number of virtual and other stimulation sites provides a great deal of flexibility with respect to positioning the neural excitation areas precisely in the cochlear place that best conveys the frequencies of the incoming sound.

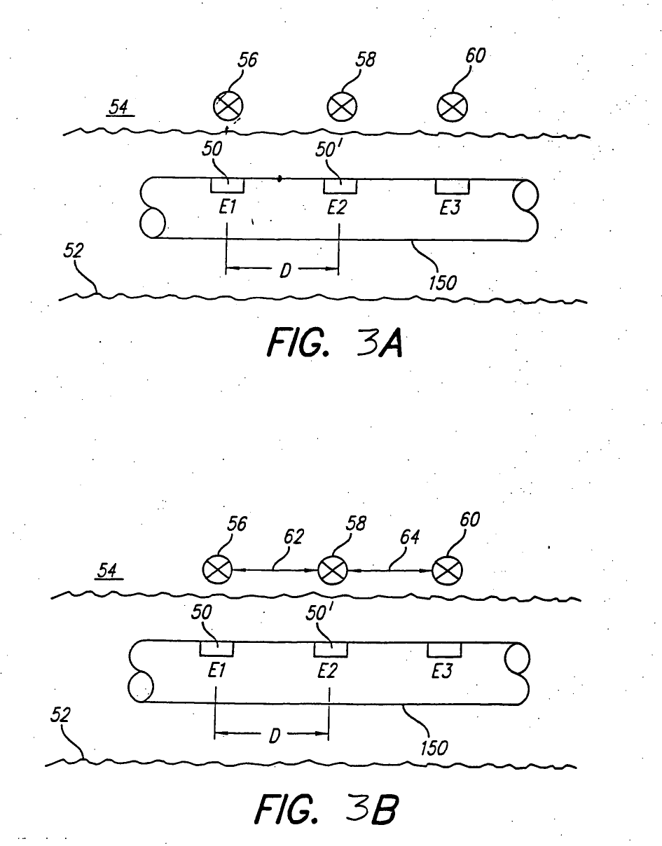

As explained in more detail below in connection with FIGS. 3A and 3B, through appropriate weighting and sharing of currents between two or more physical electrodes, it is possible to provide a large number of virtual electrodes between physical electrodes, thereby effectively steering the location at which a stimulus is applied to almost any location along the length of the electrode array.

The output stage of the ICS 21 which connects with each electrode E1, E2, E3 . . . Em of the electrode array may be as described in U.S. Pat. No. 6,181,969, incorporated herein by reference. Such output stage advantageously provides a programmable N-DAC or P-DAC (where DAC stands for digital-to-analog converter) connected to each electrode so that a programmed current may be sourced to the electrode or sunk from the electrode. Such configuration allows any electrode to be paired with any other electrode and the amplitudes of the currents can be programmed and controlled to gradually shift the stimulating current that flows from one electrode through the tissue to another adjacent electrode or electrodes, thereby providing the effect of “shifting” the current from one or more electrodes to another electrode(s). Through such current shifting, the stimulus current may be shifted or directed so that it appears to the tissue that the current is coming from or going to an almost infinite number of locations.

Next, with reference to FIG. 3A, a diagram is presented to illustrate the location where a stimulus is applied when virtual electrodes are employed. In FIG. 3A, three electrodes E1, E2 and E3 of an electrode array are illustrated. A reference electrode, not shown, is also presumed to be present some distance from the electrodes E1, E2 and E3, thereby allowing monopolar stimulation to occur between a selected one of the electrodes and the reference electrode. Bipolar stimulation could likewise occur, e.g., between electrodes E1 and E2, between E2 and E3, or between any other pair of electrodes.

The electrodes E1, E2 and E3 are located “in line” on a carrier 150, and are spaced apart from each other by a distance “D”. Each electrode is electrically connected to a wire conductor (not shown) that is embedded within the carrier 150 and which connects the electrode to the ICS 21 (see FIGS. 2A or 2B). The carrier 150 is shown inserted into a duct 52 within tissue 54 that is to be stimulated. For a cochlear implant system, the duct 52 typically comprises the scala tympani of a human cochlea.

When a stimulus current is applied to electrode E1, the stimulus location in the tissue 54 is essentially the location 56, adjacent the physical location of the electrode E1. Similarly, when a stimulus current is applied to electrode E2, the stimulus location in the tissue 54 is essentially the location 58, adjacent the physical location of the electrode E2. Likewise, when a stimulus current is applied to electrode E3, the stimulus location in the tissue 54 is essentially the location 60, adjacent the physical location of the electrode E3. It is thus seen that the resolution or precision, with which a stimulus may be applied to the tissue is only as good as is the spacing of the electrodes on the electrode array. That is, each stimulus location in the tissue 54 is separated by approximately the same distance “D” as separates the electrodes.

With reference to FIG. 3B, a diagram is presented to illustrate the location where a stimulus is applied when virtual electrodes are employed, specifically by using current steering. The structure of the electrode array and spacing between electrodes E1, E2 and E3 is the same as in FIG. 3A. Thus, when a stimulus current is applied only to electrode E1, the stimulus location in the tissue 54 is the location 56, the same as was the case in FIG. 3A. Similarly, when a stimulus current is applied only to electrode E2, the stimulus location in the tissue 54 is the location 58. Likewise, when a stimulus current is applied only to electrode E3, a stimulus location in the tissue 54 is the location 60. However, through application of current steering, a stimulus current may be shared, e.g., between electrodes E1 and E2 (and some other paired or reference electrode), and the effective tissue location where the stimulus is directed may be anywhere along the line 62 between points 56 and 58. Alternatively, if the current is shared between electrodes E2 and E3, the location in the tissue where the stimulus is directed may be anywhere along the line 64 between points 58 and 60.

To illustrate further, suppose a stimulus current having an amplitude I1 is applied to the tissue through electrode E1 (and some reference electrode). The location within the tissue 54 where the stimulus would be felt would be the point 56. However, if a stimulus current of only 0.9×I1 were applied through electrode E1 at the same time that a stimulus current of 0.1×I1 where applied through electrode E2, then the location within the tissue 54 where the stimulus would be felt would be a little to the right of the point 56, more or less somewhere on the line 62. If the stimulus current applied through electrode E1 continued to be deceased while, at the same time, the current applied through electrode E2 were increased, then the location in the tissue where the stimulus would be directed would move along the line 62 from left to right, i.e., from point 56 to point 58.

Similarly, by concurrently delivering current stimuli at electrodes E2 and E3, the location in the tissue where the effective stimulus would be felt would lie somewhere along the line 64, depending on the weighting of stimulus currents delivered at the two electrodes. This concept of current steering is described more fully in U.S. Pat. No. 6,393,325, incorporated herein by reference.

It is noted that the concept of virtual electrodes which directs a stimulus to a location on the cochlear location or place is a broad concept. One method of implementing virtual electrodes is by concurrently delivering stimuli at two or more electrodes. Another way of implementing virtual electrodes is to present alternating stimuli at two electrodes in a time-multiplexed manner. For example, a first stimulus current is presented at the first electrode, then a second stimulus current is presented at the second electrode, then the first stimulus current is presented at the first electrode, then second stimulus current is presented at the second electrode, and so on, in a time multiplexed sequence. The first and second stimulus signals are usually different, e.g., they have different amplitudes and/or pulsewidths. Such delivery of stimulation will be perceived as if a virtual electrode were delivering a stimulus, which virtual electrode appears to be located between the two physical electrodes.

As discussed, each place along the cochlea corresponds to a specific perceived sound frequency. That is, different frequencies cause maximum vibration amplitude at different points along the cochlea. Low frequency sounds create traveling waves in the fluids of the cochlea that cause the cochlea's basilar membrane to vibrate with largest amplitude of displacement at the apex of the basilar membrane. High frequency sounds create traveling waves with largest amplitude of displacement at the base of the basilar membrane. If the signal is composed of multiple frequencies, then the resulting traveling wave will create maximum displacement at different points along the basilar membrane. Pursuant to the foregoing concepts, for an electrode array implanted into the cochlea, the spatial frequency represented by each electrode contact of the electrode array must correspond to the spatial frequency or “place” along the cochlea.

Consequently, in order for the patient to properly perceive sounds with the implant, the implant must be “fitted” or “tuned” to accommodate the electrode array's particular placement in the cochlea. Such a fitting method includes a pitch ranking and channel allocation process. Pursuant to this process, the electrodes of the electrode array are ranked based on their pitch. The speech processor then assigns certain frequency bands to each electrode of the array such that each electrode is associated with a particular channel that represents a frequency or range of frequencies. An exemplary pitch ranking process is described in U.S. Provisional Patent Application Serial No. 60/313,694, filed Aug. 20, 2001, which application, including its Appendix A, is incorporated herein by reference in its entirety.

In the case of the patient having bilateral implants, the patient has a first implant (referred to herein as the primary implant) in a first ear and a second implant (referred to herein as the secondary implant) in the second ear. Pursuant to the pitch ranking process, the electrodes in the secondary implant are mapped to corresponding electrodes in the primary implant such that the electrodes in both implants represent similar frequency bands. For example, electrode 1 in the primary implant and electrode 1 in the secondary implant can both be assigned a frequency of 400 Hz. In such a case, electrode 1 in the primary implant is considered mapped to electrode 1 in the secondary implant as both electrodes are assigned similar frequency bands.

However, it is common for the primary and secondary electrode arrays to be not generally matched in location along their respective cochleas. In other words, the electrode array in one ear is often positioned at a different location along the cochlea than the electrode array in the other ear. This makes it difficult to precisely match the electrodes in one ear to corresponding electrode in the other ear. Absent the use of virtual electrodes, the matching of electrodes between bilateral implants can only be accomplished within the precision of the electrode spacing in the array.

There is now described a process for more exactly matching the pitch allocation between the electrode arrays in opposite ears. The described method takes advantage of the concept of virtual electrodes, which enable the channel of an electrode in one ear to be mapped to a synthetic channel (i.e., a virtual electrode) in another ear. For example, it can be determined that the patient's perceived frequency when the cochlear place of the primary implant's electrode 1 is stimulated does not match the perceived frequency when the cochlear place of the secondary implant's electrode 1 is stimulated. Rather, the frequency of electrode 1 actually corresponds to the frequency of a virtual electrode positioned somewhere in between electrode 1 and electrode 2. In such a case, the primary implant's electrode 1 is mapped to a virtual electrode in the secondary implant.

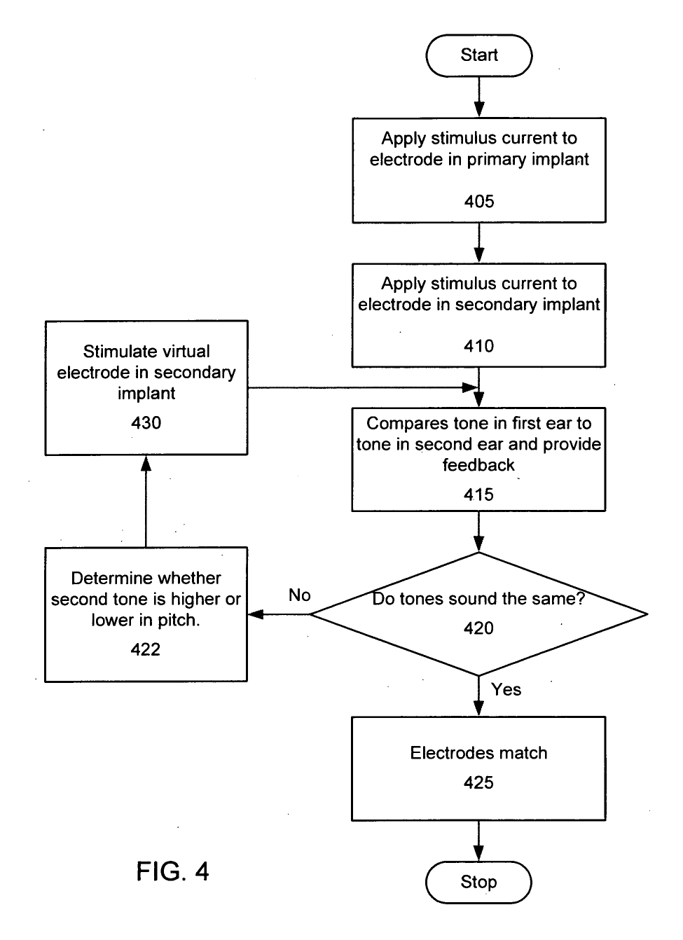

This is described in more detail with reference to FIG. 4, which shows a flow diagram that illustrates an exemplary method of pitch matching an electrode in one implant to an electrode in another implant. Each step in the method shown in FIG. 4 is summarized in a block. The relationship between the steps i.e., the order in which the steps are carried out, is represented by the manner in which the blocks are connected in the flow chart. Each block has a reference number assigned to it.

In a first operation, represented by flow diagram box 405, a stimulus current is applied to a predetermined electrode in the primary implant. For example, assume that the electrode array in FIG. 3A is the electrode array of the primary implant. A stimulus current is applied, for example, to electrode E1, which corresponds to stimulus location 56. The stimulation of electrode E1 results in the patient's first ear perceiving a tone of the frequency associated with cochlear location 56. Thus, a first pitch of predetermined frequency is generated in a first ear using a predetermined electrode, such as electrode E1. This can also be accomplished by playing a tone in the patient's first ear, the tone having a frequency that is the same frequency as the frequency mapped to electrode E1. This would result in the processor applying a current stimulus to electrode E1.

In the next operation, represented by flow diagram box 410, a stimulus current is applied to the electrode in the secondary implant that is mapped to the previously-stimulated electrode in the primary implant (such that the electrode in the secondary was allocated the same frequency as the previously-stimulated electrode in the primary implant). For example, assume that FIG. 3B shows the electrode array of the secondary implant. Further assume that electrode E1 in the secondary implant of FIG. 3B was allocated the same frequency as electrode E1 in the primary implant of FIG. 3A. In such a case, a stimulus current is applied to electrode E1 in the secondary implant. This will result in the patient's second ear perceiving a tone of a certain frequency wherein the frequency is that frequency actually associated with cochlear location 56. Note that, due to differences in cochlear placement of the primary and secondary implants, the actual pitch associated with cochlear place 56 (FIG. 3A) in the primary implant may not be the same as the actual pitch of the cochlear place 56 (FIG. 3B) in the secondary implant.

It should be appreciated that the operations represented by flow diagram boxes 405 and 410 can be performed concurrently or sequentially in any given order. For example, the electrode in the primary implant can be stimulated first and then the electrode in the secondary implant stimulated after stimulation of the primary electrode is ceased, or vice-versa. This would result in the patient first hearing a tone in the first ear and then hearing a tone in the second ear after the tone in the first ear ceases. Alternately, the electrodes in the two ears can be stimulated simultaneously such that the patient simultaneously hears a tone in both ears. In any event, the electrodes can be stimulated in the manner that best permits the patient to compare and contrast the tones in the first and second ears.

With reference still to the flow diagram of FIG. 4, the next operation is represented by the flow diagram box 415, where the patient compares the tone perceived in the first ear to the tone perceived in the second ear and further provides feedback regarding the comparison. In this regard, the patient provides information as to whether the tone in the first ear appears to have the same pitch as the tone in the second ear. The patient can also provide information as to whether he or she is hearing a unified sound sensation in both ears or whether the sensations appear to be different from one ear to the other ear. Essentially, the patient provides feedback as to whether the tones in the two ears “sound the same.”

The next operation is represented by decision box 420 in FIG. 4, where the patient decides whether the tones in the two ears sound the same. If the patient indicates that the tones in the two ears do sound the same (a “Yes” output from decision box 420), then this indicates that the stimulated electrode in the primary implant is properly pitch matched to the stimulated electrode in the secondary implant, as represented by flow diagram box 425. In other words, stimulation of the electrode in the primary implant resulted in the patient perceiving a tone in the first ear of the same frequency as a tone in the second ear resulting from stimulation of the electrode in the secondary implant. This indicates that the electrode arrays in the two ears are aligned along their respective cochlea.

However, if the patient indicates that the tones in the two ears do not sound the same (a “No” output from decision box 420 in FIG. 4), then this indicates that the electrodes in the first ear are not pitch matched to the electrodes in the second ear. In other words, stimulation of the electrode in the first ear results in the patient perceiving a tone of a different frequency than the tone perceived when the corresponding electrode is stimulated in the second ear.

The process then proceeds to the operation represented in flow diagram box 422 in FIG. 4, where it is determined whether the tone in the second ear sounds higher in pitch or lower in pitch than the tone in the first ear. The process then proceeds to the operation represented in the flow diagram box 430 in FIG. 4. In this operation, for the secondary implant, a virtual electrode is stimulated at a location adjacent the physical electrode that was previously stimulated, while maintaining the stimulation of the same electrode in the primary implant. The initial location of the virtual electrode is based on whether the tone in the second ear sounded higher or lower in pitch than the tone in the first ear. In this manner, the patient will perceive a tone of a different frequency (corresponding to the frequency of the cochlear place of the virtual electrode) in the second ear.

For example, assume that FIG. 3A shows the electrode array in the second ear and that electrode E1 was previously stimulated. In this operation, a virtual electrode somewhere along the line 62 is stimulated or somewhere along the opposite side of electrode E1, which will result in the patient's second ear perceiving a tone of a different frequency than was perceived when electrode E1 was stimulated. The goal is to vary the location of the virtual electrode so as to stimulate a cochlear place in the second ear's cochlea such that the patient's second ear perceives a tone of the same frequency as perceived in the first ear.

The process then returns to the operations of boxes 415 and 420, where the patient provides feedback as to the pitches of the tones perceived in both ears and decides whether the tones sound the same. The location of the virtual electrode in the second ear is iteratively varied in combination with patient feedback, until the patient determines that the tones in the two ears sound the same. It should be appreciated that the manner in which the virtual electrode is stimulated and the manner in which the location of the virtual electrode is varied can vary widely. For example, the location of the virtual electrode can be varied in a discrete manner such that a virtual electrode at first location is stimulated, patient feedback is obtained, a virtual electrode at a second location is stimulated, patient feedback is obtained, and so on until the tones in both ear match. Alternately, the location of the virtual electrode can be varied along a continuum, such as by providing an operator with an input device, such as a knob or a graphical user interface, that permits the operator to continuously vary the current steering between one electrode and an adjacent electrode and thereby continuously vary the location of the virtual electrode along a continuum. Thus, with reference to FIG. 3B, the operator would effectively “slide” the location of the virtual electrode along line 62 (or along a line on the opposite side of electrode 56) until the two tones sound the same.

As discussed above, the stimulation of the virtual electrodes can be implemented in various manners, such as using current steering between two or more electrodes or by presenting alternating stimuli at two electrodes in a time-multiplexed manner.

When the patient determines that the tone in the first ear (resulting from stimulation of a physical electrode) sounds the same as the tone in the second ear (resulting from stimulation of a virtual electrode), a “yes” output results from decision box 420. The process then proceeds to the operation of flow box 425. In this case, the primary electrode matches a virtual electrode in the secondary implant. This process can be repeated for additional electrodes as desired.

If the foregoing process determined that the physical electrodes in the primary implant do not match the physical electrodes in the secondary implant, then the mapping of electrodes from the first implant to the second implant can be adjusted accordingly. For example, one or more electrodes in the primary implant can be mapped to one or more virtual electrodes in the secondary implant. For example, electrode E1 of the primary implant shown in FIG. 3A can be mapped to a virtual electrode located somewhere along the line 62 of the secondary implant in FIG. 3B. This can be accomplished, for example, by adjusting the mapping circuitry 41 and/or the pulse table (shown in FIG. 2B) such that the frequency associated with the electrode in the primary implant is associated with a virtual location via a joint map link . Thus, a signal that results from analysis in one of the analysis channels will be jointly mapped to two simulation channels to form a virtual simulation channel of the virtual electrode.

Alternately, the configuration of the band pass filters 30 (shown in FIG. 2B) of the secondary implant can be modified to adjust the center frequency to compensate for the offset in alignment between the cochlear placement of an electrode in the primary implant relative to an electrode in the secondary implant. This might be easier understood in the context of an example. Assume that it is determined that, for the primary implant, electrode E1 maps to a frequency of 350 Hz and electrode E2 maps to a frequency of 450 Hz. It is then determined, based on the process described above, that primary electrode E1 maps to a virtual electrode that is halfway between electrode E1 and E2 in the secondary implant (such a virtual electrode is referred to herein as “electrode 1.5”). This means that the virtual electrode 1.5 should be mapped to a frequency of 350 Hz, which is the same frequency mapped to electrode E1 in the primary implant. In such a case, the center frequency of the band pass filter for electrode E1 in the secondary implant can be set to 300 Hz and the center frequency for the band pass filter of secondary electrode E2 can be set to 400 Hz, which effectively maps virtual electrode 1.5 to a frequency of 350 Hz, which is the same frequency of electrode E1 in the primary implant.

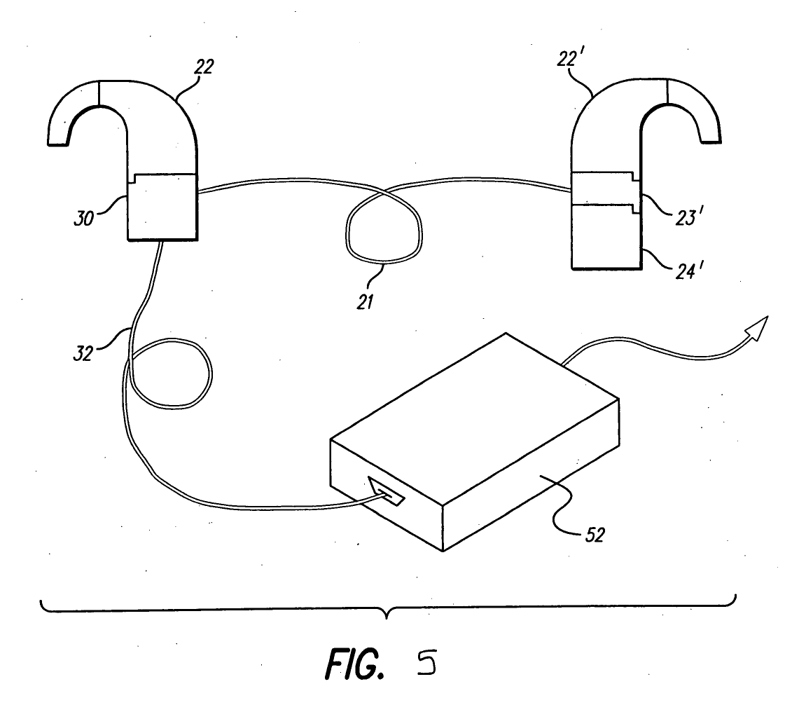

As mentioned, the primary and secondary BTEs and their associated implants can be synchronized during a fitting or programming process. FIG. 5 shows a system configuration that can be used to map primary and secondary BTEs. The BTEs are each equipped with a communications interposer, which is a device that enables communication between the BTEs and a clinician's programming interface (CPI). The CPI is a special interface unit that allows the clinician's programmer (usually a laptop computer) to interface with the BTE processor that is being programmed.

With reference to FIG. 5, a secondary or slave BTE 22′ is connected through, e.g., a first interposer 23′ and a synchronous binaural interface cable 21 to an interposer 30. The interposer 30 is connected to a primary or master BTE 22. The binaural fitting cable 32 that exits from the interposer 30 is connected to a CPI device 52. The CPI device 52, in turn, is connected to a host programming system, e.g., a laptop computer (not shown) loaded with the appropriate fitting software.

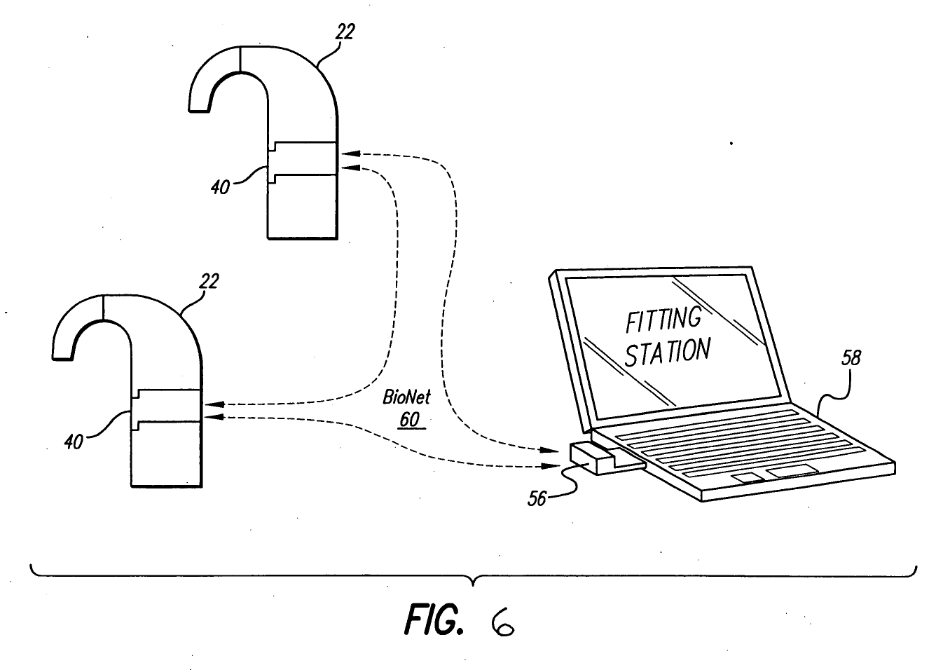

FIG. 6 shows a wireless fitting system. FIG. 11 embodies the operational modes for fitting and operating a wireless BTE fitting system. As seen in FIG. 11, the system consists of two interposers 40, each connected to a respective BTE 22, and a BioNet PC Card 56 plugged into a host fitting station 58. As thus configured, a BioNet 60 is created that allows either BTE to be coupled to the host fitting station 58, and that further allows either BTE to be coupled to the other BTE. Co-pending U.S. patent application Ser. No. 10/218,615, entitled “Bionet for Bilateral Cochlear Implant Systems”, incorporated herein by reference, describes various configurations and components of systems for allowing bilateral implant systems to be networked together.

A number of embodiments have been described. Nevertheless, it will be understood that various modifications may be made without departing from the spirit and scope of the claims. Accordingly, other embodiments are within the scope of the following claims.

What is claimed is:

1. A method of pitch matching a first cochlear implant to a second cochlear implant, the method comprising:
applying a first stimulus current to a first electrode of a first multi-electrode array implanted in a first cochlea of a user to generate a first tone relative to a first ear of the user;
applying a second stimulus current to a second electrode of a second multi-electrode array in a second cochlea of the user to generate a second tone relative to a second ear of the user;
obtaining feedback as to whether the first tone matches the second tone;
if it is determined that the first tone does not match the second tone, applying a stimulus current to a virtual electrode of the second multi-electrode array, wherein stimulation of the virtual electrode generates a third tone that matches the first tone; and
mapping a position of the first electrode of the first multi-electrode array to a position corresponding to the virtual electrode of the second multi-electrode array.

2. A method as defined in claim 1, wherein mapping the first electrode of the first multi-electrode array to the virtual electrode of the second multi-electrode array comprises allocating a frequency of a received sound signal to both the first electrode and to the virtual electrode.

3. A method as defined in claim 1, further comprising continuously varying the cochlear location of the virtual electrode to thereby vary the perceived frequency of the third tone until it is determined that the third tone matches the first tone.

4. A method as defined in claim 1, wherein obtaining feedback comprises obtaining information from the user as to whether the first tone appears to be of the same pitch as the second tone.

5. A method as defined in claim 1, wherein obtaining feedback comprises obtaining information from the user as to whether the first tone sounds the same as the second tone.

6. A method as defined in claim 1, wherein applying a stimulus current to the virtual electrode comprises presenting weighted stimulus currents simultaneously at two electrodes in the second multi-electrode array.

7. A method as defined in claim 1, wherein applying a stimulus current to the virtual electrode comprises presenting rapidly alternating stimulus currents at two closely spaced electrodes in the second multi-electrode array in a time-multiplexed manner.

8. A method as defined in claim 1, further comprising performing the method for a plurality of electrodes in the first multi-electrode array and a plurality of corresponding electrodes in the second multi-electrode array.

9. A method of matching a first cochlear implant to a second cochlear implant, the method comprising:
applying a first stimulus current to a first electrode of a first multi-electrode array implanted in a first cochlea of a user to generate a first tone in a first ear of the user;
determining the cochlear position of a first virtual electrode of a second multi-electrode array implanted in a second cochlea of the user, wherein stimulation of the virtual electrode results in a second tone in the second ear of the user that matches the first tone; and
mapping the first electrode to the first virtual electrode.

10. A method as defined in claim 9, wherein determining the location of a first virtual electrode comprises continuously varying the cochlear position of the first virtual electrode until the first tone matches the second tone.

11. A method as defined in claim 9, wherein stimulation of the first virtual electrode is accomplished by presenting weighted stimulus currents simultaneously at two electrodes in the second multi-electrode array.

12. A method as defined in claim 9, wherein stimulation of the first virtual electrode is accomplished by presenting rapidly alternating stimulus currents at two closely spaced electrodes in the second multi-electrode array in a time-multiplexed manner.

13. A method as defined in claim 9, wherein mapping the first electrode of the first multi-electrode array to the first virtual electrode of the second multi-electrode array comprises allocating a frequency of a received sound signal to both the first electrode and to the first virtual electrode.

14. A method as defined in claim 9, further comprising:
applying a stimulus current to a second electrode of the first multi-electrode array implanted in a first cochlea of a user to generate a third tone in a first ear of the user;
determining the cochlear position of a second virtual electrode of the second multi-electrode array implanted in a second cochlea of the user, wherein stimulation of the virtual electrode results in a fourth tone in the second ear of the user that matches the third tone; and
mapping the second electrode to the second virtual electrode.

15. A method as defined in claim 9, further comprising mapping multiple electrodes in the first multi-electrode array to multiple virtual electrodes in the second multi-electrode array.

16. A method as defined in claim 9, wherein determining the cochlear position of a first virtual electrode of a second multi-electrode array implanted in a second cochlea of the user comprises:
varying the cochlear location of the first virtual electrode to vary the perceived frequency of the second tone;
fixing the cochlear location of the first virtual electrode when the perceived frequency of the second tone is the same as the perceived frequency of the first tone.

17. A bilateral cochlear stimulation system, comprising:
a first cochlear implant system comprising first multi-electrode array having a first plurality of electrodes configured for placement in first cochlear duct of a patient;
a second cochlear implant system comprising a second multi-electrode array having a second plurality of electrodes configured for placement in a second cochlear duct of a patient, the second multi-electrode array being configured to implement virtual electrodes;
wherein a first electrode of the first multi-electrode array is mapped to a virtual electrode of the second multi-electrode array.

18. A bilateral cochlear stimulation system as defined in claim 17, wherein multiple electrodes of the first multi-electrode array are mapped to multiple virtual electrodes of the second multi-electrode array

19. A bilateral cochlear stimulation system as defined in claim 17, wherein the first and second cochlear implant systems each further comprise:
an acoustic transducer for sensing acoustic signals and converting them to electrical signals;
analog front end circuitry for preliminarily processing the electrical signals produced by the acoustic transducer;
an implantable cochlear stimulator connected to a respective first or second electrode array for generating electrical stimuli defined by control signals; and
a speech processor that generates the control signals used by the ICS.

20. A bilateral cochlear stimulation system as defined in claim 17, wherein each cochlear implant system includes a behind the ear (BTE) unit positionable near the patient's ear, each BTE unit comprising a respective speech processor, a microphone, and a battery unit.

21. A bilateral cochlear stimulation system as defined in claim 17, wherein each cochlear implant system includes an interposer module, wherein the interposer modules enable communication between the first cochlear implant system and the second cochlear implant system.
